(19) United States
(12) Patent Application Publication
(10) Pub. No.: US 2026/0306293 A1
(43) Pub. Date: Oct. 8, 2026

(54) HEAT TRANSFER-BASED MEDICAL TREATMENT SYSTEMS, METHODS, AND DEVICES

(71) Applicant: THE BOARD OF REGENTS OF THE UNIVERSITY OF TEXAS SYSTEM, Austin, TX (US)

(72) Inventor: Syed Faaiz Enam, Dallas, TX (US)

(21) Appl. No.: 19/485,971

(22) PCT Filed: May 24, 2024
(86) PCT No.: PCT/US2024/031118

§ 371 Date: Nov. 19, 2025

Related U.S. Application Data
(60) Provisional application No. 63/504,196, filed on May 24, 2023.
(60) Provisional application No. 63/579,504, filed on Aug. 29, 2023.
(60) Provisional application No. 63/644,375, filed on May 8, 2024.
(60) Provisional application No. 63/644,389, filed on May 8, 2024.

(51) Int. Cl. A61F 7/00

(52) U.S. Cl. CPC A61F 7/007; A61F 2007/0002; A61F 2007/0056; A61F 2007/0075

(57) ABSTRACT

Systems, methods, and devices include a heat transfer treatment device which uses hypothermia to slow and/or halt brain tumor growth (e.g., “cytostatic hypothermia”). These fully and/or partially implantable device(s) use a physics-based approach to treat the targeted cells in a patient. The heat transfer treatment device includes a body portion having a top surface and a bottom surface. A cooling probe array with a particular probe geometry design includes a plurality of cooling probes extending at least partly from the bottom surface. The heat transfer treatment device also includes an injection delivery system having a port, a reservoir fluidly coupled to the port, and/or a delivery tunnel fluidly coupled to the reservoir and extending through the body portion out the bottom surface. As such, the heat transfer treatment device provides hypothermia treatment concomitantly with chemotherapy and immunotherapy.

### CROSS-REFERENCE TO RELATED APPLICATIONS

The present application claims priority to U.S. Provisional Application No. 63/504,196 filed May 24, 2023, U.S. Provisional Application No. 63/644,375 filed May 8,2024, U.S. Provisional Application No. 63/579,504 filed Aug. 29, 2023, and U.S. Provisional Application No. 63/644,389 filed May 8, 2024. Each of these applications is incorporated by reference in its entirety herein.

### BACKGROUND

There are a variety of available techniques for treating brain tumors such as radiation, chemotherapy, and surgery. However, each patient has a unique growth or geometry of their brain tumor. In addition, with surgical resection or any other treatment, the resulting anatomy is also unique. To appropriately target unique geometries and situations, a portfolio of options is required that can be tailored to the patient.

It is with these observations in mind, among others, that various aspects of the presently disclosed technology were conceived and developed.

### BRIEF SUMMARY

The systems, methods, and devices disclosed herein address the aforementioned problems. For instance, a tumor treatment device can include a body portion having a top surface and a bottom surface and can also include a cooling probe array with a probe geometry design such that a plurality of cooling probes extends at least partly from the bottom surface. Also, an injection delivery system can be operable to provide an injectable substance through the body portion to a target area. Furthermore, the body portion can include a water block layer defining the top surface. Additionally, the injection delivery system can include a reservoir operable to hold the injectable substance being provided to the target area. The reservoir can be formed on the water block layer at the top surface and/or can be centrally located on the top surface.

In some instances, the injection delivery system can include a delivery tunnel passing at least partly through the body portion to provide the injectable substance to the target area. For example, the delivery tunnel can extend out the bottom surface and/or can include an extension portion protruding from the bottom surface between the plurality of cooling probes. Also, the reservoir and/or the delivery tunnel can be formed of biocompatible material. Additionally, the injection delivery system can include a port disposed at the top surface operable to receive a needle providing the injectable substance. The port can be fluidly coupled to the delivery tunnel and/or the reservoir. Moreover, the body portion can include a Peltier array layer disposed between the water block layer and the cooling probe array.

In some scenarios, a tumor treatment system can include a body portion of a tumor treatment device having a top surface and a bottom surface. The tumor treatment system can also include a cooling probe array of the tumor treatment device having a probe geometry design such that a plurality of cooling probes extends at least partly from the bottom surface. The cooling probe array can be operable to cool a target area of a patient. Also, an injection delivery system of the tumor treatment system can be operable to provide an injectable substance through the body portion. The injection delivery system can include a port operable to receive the injectable substance; a reservoir fluidly coupled to the port; and/or a delivery tunnel fluidly coupled to the reservoir and extending through the body portion to the target area. Furthermore, the body portion can be operable for placement at an interior wall of a blood vessel; and/or the target area can include a plasma flow path within the blood vessel.

In some examples, a method of tumor treatment includes positioning a body portion of a tumor treatment device on an external surface of a patient; and/or inserting a plurality of cooling probes of a cooling probe array into the patient at a target area. The target area can be below the external surface of the patient, and the plurality of cooling probes can have a probe geometry design such that the plurality of cooling probes extends at least partly from a bottom surface of the body portion of the tumor treatment device. The method can also include treating a tumor of the patient using the tumor treatment device by cooling the target area with the plurality of cooling probes. Treating the tumor of the patient can further include injecting a pharmaceutical into a port disposed at a top surface of the body portion of the tumor treatment device such that the pharmaceutical passes through the body portion to the target area via a delivery tunnel. Also, treating the tumor of the patient can include transferring a therapeutic from a reservoir disposed in the body portion to the target area simultaneously with cooling the target area. Additionally, cooling the target area with the plurality of cooling probes can include providing cooling water to a water block layer of the body portion. The water block layer can be disposed adjacent to a Peltier array layer of the body portion.

In some examples, a tumor treatment device can include a body portion having a top surface and a bottom surface. The body portion can include a water block layer; and/or a Peltier array disposed adjacent to the water block layer. Also, the tumor treatment device can include a cooling probe array with a probe geometry design such that a plurality of cooling probes extends at least partly from the bottom surface. Additionally, the probe geometry design can include a probe mounting platform, and at least some of the plurality of cooling probes can extend perpendicularly from the probe mounting platform. Moreover, the probe geometry design can include at least a core-and-spoke arrangement of the plurality of cooling probes. The probe geometry design can also include a plurality of curved probes forming a splayed arrangement. Furthermore, the probe geometry design can include a patch with a removable structural support. The removable structural support can have a first configuration as a layer on the top surface and a second configuration removed from the top surface. An injection delivery system can be exposed upon removing the removable structural support.

In some examples, the probe geometry design can include a multi-platform arrangement. This multi-platform arrangement can include at least: first probe mounting platform operable for placement on an outer cranial surface; and/or a second probe mounting platform operable for placement under the outer cranial surface. Furthermore, the first probe mounting platform can be coupled to the second probe mounting platform by a platform extension arm. The platform extension arm can couple to a bottom surface of the first probe mounting platform and a top surface of the second probe mounting platform. Also, the plurality of cooling probes can include one or more first cooling probes extending from the first probe mounting platform and/or one or more second cooling probes extending form the second probe mounting platform. Additionally, the water block layer can define the top surface of the body portion. Moreover, the Peltier array can cause a heat transfer between the water block layer and the cooling probe array.

In some examples, a tumor treatment system can include a body portion of a tumor treatment device with a top surface defined by a water-cooling block layer. The tumor treatment system can also include a cooling probe array of the tumor treatment device having a probe geometry design such that a plurality of cooling probes extends at least partly from a bottom surface of the body portion. The probe geometry design can be operable to cool a target area of a patient. The probe geometry design can include at least one of a core-and-spoke arrangement; a splayed arrangement; a multi-platform arrangement; and/or a removable structural support. Furthermore, the tumor treatment system can include a probe implantation device having a release sheath operable to maintain the plurality of cooling probes in a first position and release the plurality of cooling probes into a second position. The first position can be a bundled position and the second position can be a splayed position.

In some scenarios, a method of tumor treatment includes positioning a body portion of a tumor treatment device on an external surface of a patient. The method can also include inserting a plurality of cooling probes of a cooling probe array into the patient at a target area, the target area being below the external surface of the patient. By way of example, the plurality of cooling probes can have a probe geometry design including at least one of a core-and-spoke arrangement; a splayed arrangement; or a multi-platform arrangement. Also, the method can include treating a tumor of the patient using the tumor treatment device by cooling the target area with the plurality of cooling probes. Furthermore, inserting the plurality of cooling probes into the patient can include inserting a probe implantation device into the patient; and/or sliding a release sheath of the probe implantation device out of the patient. Additionally, positioning the body portion of the tumor treatment device can include securing the body portion to the external surface of the patient using a removal structural support disposed on a top surface of the body portion; and/or removing the removal structural support from the body portion.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the disclosed subject matter. It should be understood, however, that the disclosed subject matter is not limited to the precise implementations and features shown. The accompanying drawings, which are incorporated in and constitute a part of this specification, illustrate implementations of systems, methods, and devices consistent with the disclosed subject matter and, together with the description, serves to explain advantages and principles consistent with the disclosed subject matter, in which:

FIG. 1A illustrates an elevation view of an example system for treating tumors with a heat transfer treatment device;

FIG. 1B illustrates a top view of an example system for treating tumors with a heat transfer treatment device;

FIG. 2A illustrates an example system for using a heat transfer treatment device having a probe geometry design;

FIG. 2B illustrates an example system for using a heat transfer treatment device having a probe geometry design including a core-and-spoke arrangement;

FIG. 2C illustrates an example system for using a heat transfer treatment device having a probe geometry design including a multi-platform arrangement;

FIG. 2D illustrates an example system for using a heat transfer treatment device having a probe geometry design including a splayed probe arrangement;

FIG. 2E illustrates an example system for using a heat transfer treatment device having a probe geometry design including a removal structural support;

FIG. 3 illustrates an example system for using a heat transfer treatment device with a slidable implantation device;

FIG. 4 illustrates an example system for using a heat transfer treatment device in a blood vessel and/or with an artificial internal circulation system; and

FIG. 5 illustrates an example method of treating a patient with a heat transfer treatment device.

FIG. 6 illustrates another example method of treating a patient with a heat transfer treatment device.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the examples described herein. However, it will be understood by those of ordinary skill in the art that the implementations described herein can be practiced without these specific details. In other instances, methods, procedures, and components have not been described in detail so as not to obscure the related relevant feature being described. Also, the description is not to be considered as limiting the scope of the implementations described herein. The drawings are not necessarily to scale, and the proportions of certain parts may be exaggerated to better illustrate details and features of the present technological concepts.

The systems, methods, and devices disclosed herein can use heat transfer techniques to induce hypothermia which slows and/or halts brain tumor growth (“cytostatic hypothermia”). The hypothermia provided can function to supplement chemotherapy and immunotherapies because, in some scenarios, chemotherapy only kills cells sensitive to the chemotherapy drugs. The fully or partially implantable device(s) disclosed herein can use a physics-based approach to treat the targeted cells in a patient regardless of the cells' sensitivity to chemotherapy drugs. In other words, a hypothermia treatment can be provided concomitantly with chemotherapy and immunotherapy. Although some systems for delivering chemotherapy or immunotherapy use intravenous injection of the therapeutic, the benefits of such therapeutics can be limited in scenarios involving brain tumors because of the blood brain barrier and/or the local constriction of blood vessels around the tumor site. Both of these effects can reduce the amount of therapeutic reaching the tumor bed. To overcome these limitations, the intracranial implant disclosed herein can be designed to provide local delivery of the therapeutics in addition to a heat transfer treatment. As a hypothermia treatment may induce vascular constriction and thus reduce delivery of chemotherapy and/or immunotherapy drugs from the intravenous route, the hypothermia treatment may be applied through an intracranial route.

For example, systems, methods, and devices disclosed herein can include a device that provides simultaneous intracranial hypothermia treatment (e.g., for treating brain tumors) and therapeutic/pharmaceutical local delivery (e.g., chemo/immunotherapy against brain tumors). This can be done with a multi-probe cooling system and a port in the device without compromising the efficacy of the hypothermia. Furthermore, multiple probe geometries and styles to enable application of therapy against brain tumors and other intracranial applications and extracranial applications are disclosed. These geometries can interface with a thermal system to cool and/or apply heat. The disclosed technology can cool cells to a target temperature (e.g., 25° C. or 20° C.) which can stop tumor growth and/or kill tumor cells. In addition to, or alternatively to killing targeted cells, the cooling can reduce the calipers of blood vessels providing blood to cells.

Additional advantages of the systems, methods, and devices disclosed herein will become apparent from the detailed description below.

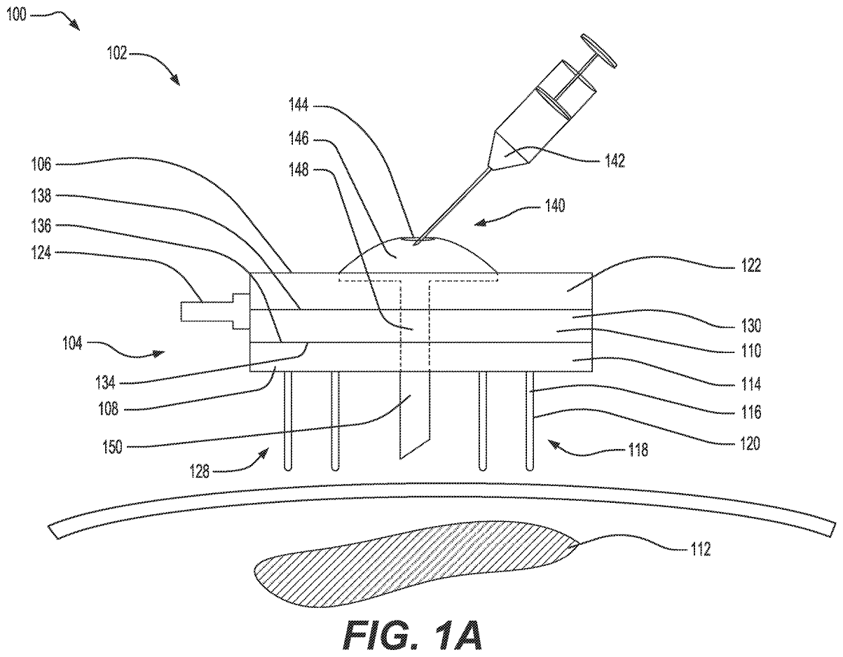

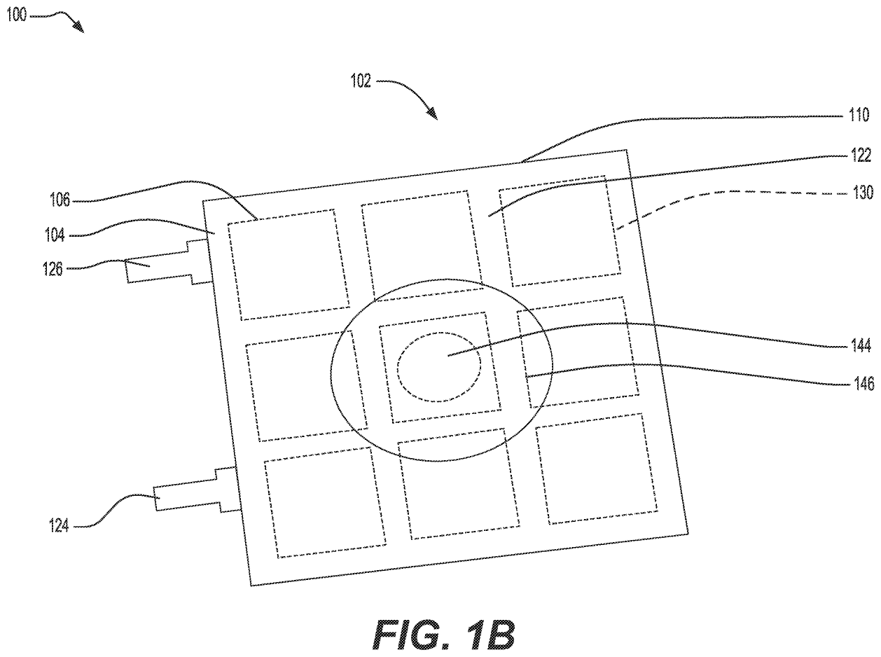

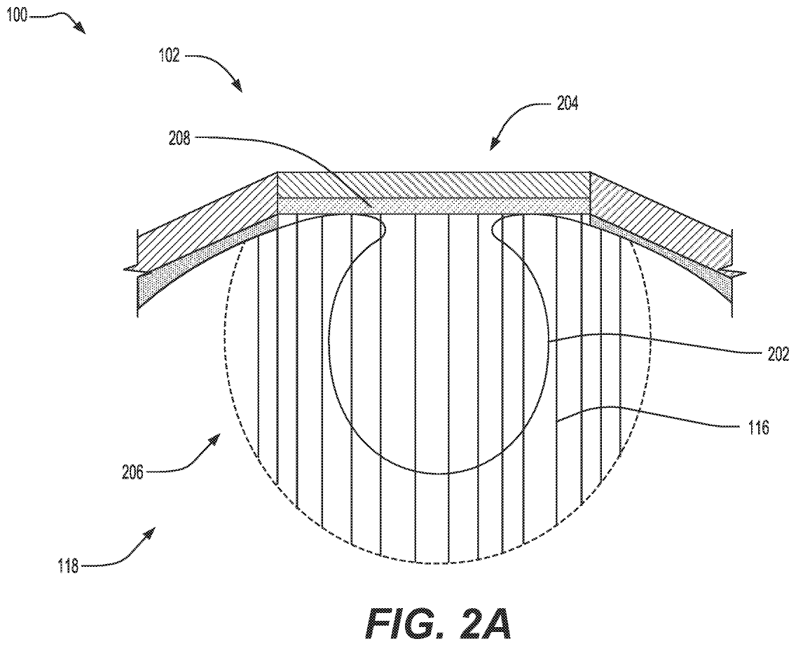

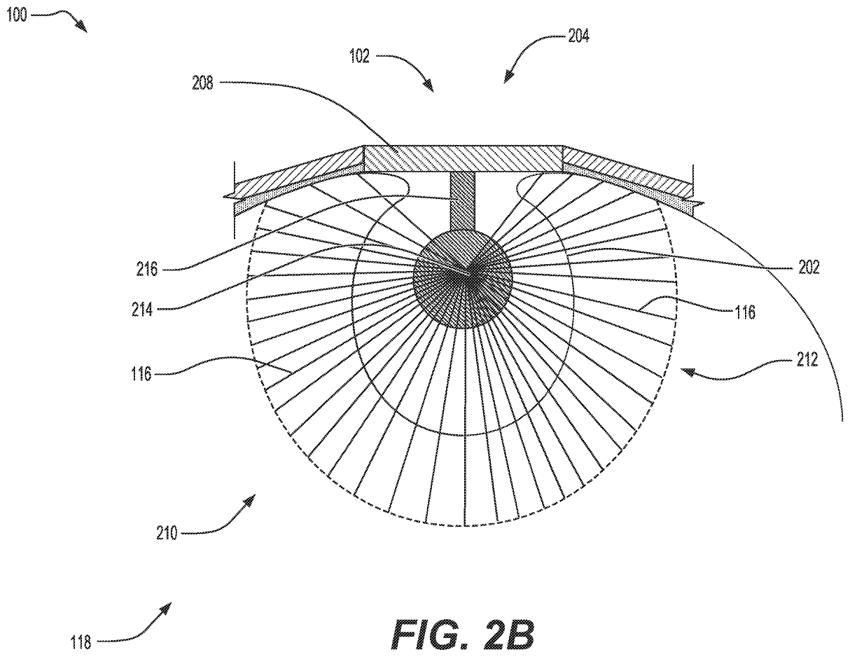

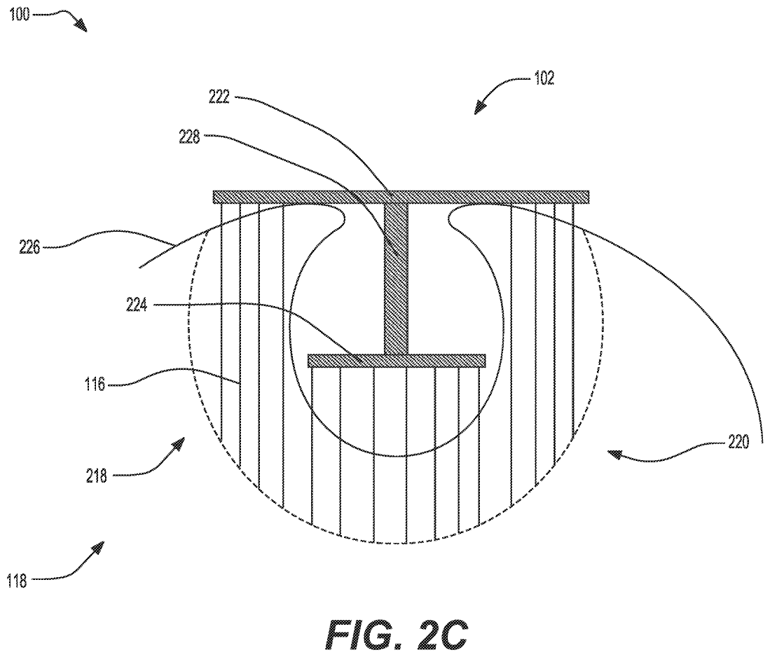

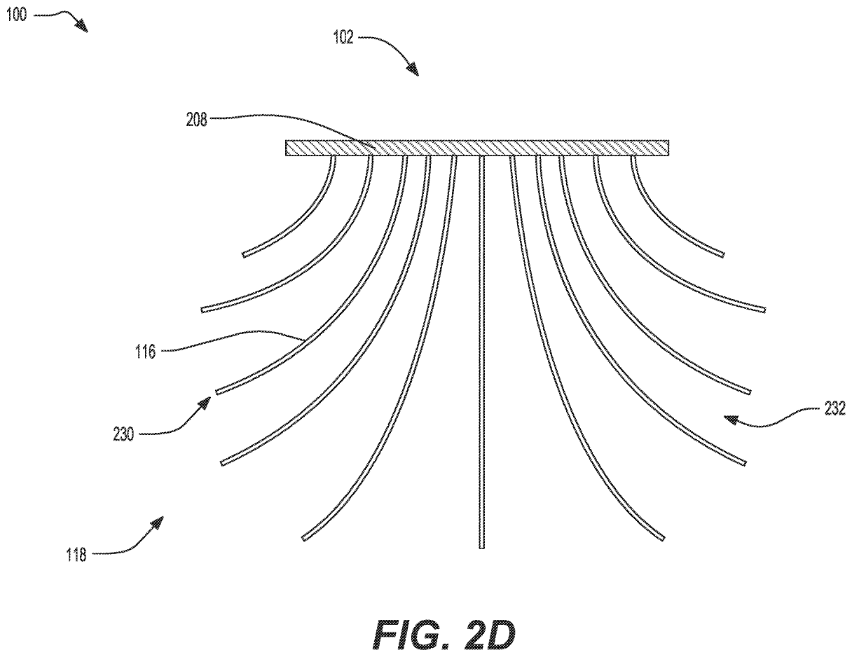

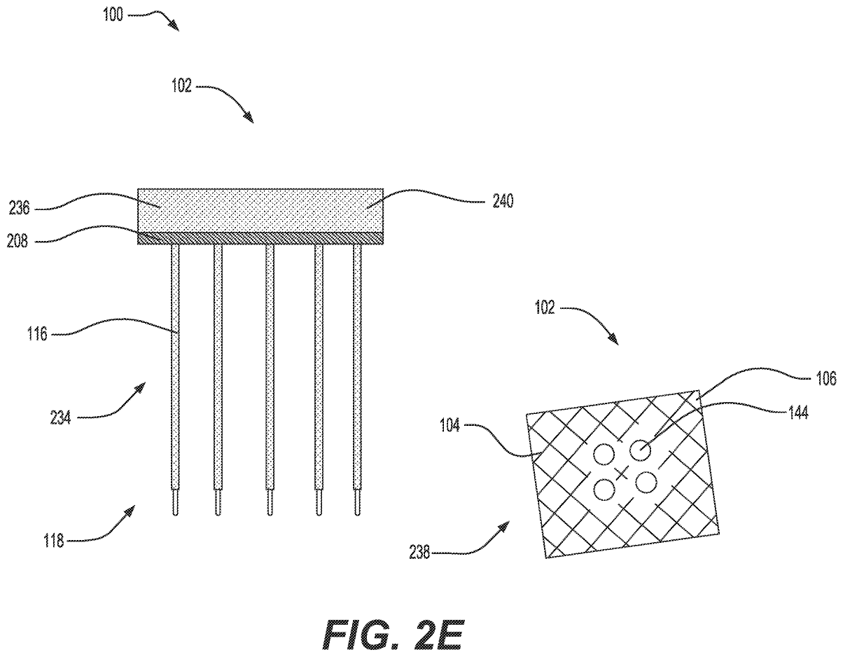

FIGS. 1A and 1B illustrate an example system 100 for providing a medical treatment, such as treating tumors (e.g., brain tumors), with a heat transfer treatment device 102. FIG. 1A shows an elevation side view and FIG. 2B shows a top view.

In some examples, the heat transfer treatment device 102 can include a body portion 104 having a top surface 106, a bottom surface 108, and one or more side surfaces 110. The body portion 104 can have a substantially square shape, circular shape, or rectangular shape formed by the side surfaces 110. Additionally, or alternatively, the body portion 104 can have a circular shape, a triangular shape, an irregular shape, and/or combinations thereof, such that the body portion 104 is operable to provide cooling to a particular target area 112. In some instances, the system(s) 100 disclosed herein can be designed, shaped, and/or scaled for use with humans, non-human species, such as rodents, pigs, or other species. Furthermore, in some examples, the patch can be applied to bionic devices (e.g., implants, artificial retina, implanted computers, actuators, motors, pumps, and so forth).

In some instances, the heat transfer treatment device 102 can include a cooling multi-probe array 114 for distributing a lower temperature gradient to the target region via conductive heat transfer. The cooling multi-probe array 114 can include a plurality of cooling probes 116 arranged with a probe geometry design 118 to cool the target area 112 via a distribution of cooled probe surface area 120 in and/or around the target area 112. The surface area 120 of the plurality of cooling can be cooled by transferring heat to a water block layer 122 of the body portion 104. For instance, the top surface 106 can be defined by a top layer being the water block layer 122. The water block layer 122 can include a cooling water input 124 and a cooling water output 126 (e.g., one or more openings, valves, nozzles, and/or combinations thereof). The water block layer 122 may be in fluid communication with a water block reservoir through one or more tubes. The water block reservoir may store a cooling fluid, such as water, on or near the patient's skin to and store body temperature fluid. One or more pumps may pump the body temperature fluid to the water block layer 122 of the device 102 for cooling of the “hot end” of the probes, as described in more detail below. In some instances, a cooling system 128 of the heat transfer treatment device 102 can include a Peltier array 130, which can be disposed below the water block layer 122 and/or between the water block layer 122 and the plurality of cooling probes 116. The Peltier array 130 can create a heat flux between the water block layer 122 and the multi-probe array 114 disposed at a bottom surface 134 (e.g., a surface opposite the water block layer 122) of the Peltier array 130, for instance, responsive to receiving an electrical current via an electrical connector. A cooling side 136 of the Peltier array 130 can be the bottom surface 134 to which the plurality of cooling probes 116 attach, and a heated side 138 of the Peltier array 130 can be the other surface to which the water block layer 122 attaches. The plurality of cooling probes 116 can include metal, ceramic, or other conducting material formed into elongated protrusions (e.g., pins, threads, rods, etc.). Additionally, in some scenarios, heating may be applied to the target area 112 rather than cooling, in which case the heated side 138 and the cooling side 136 of the Peltier array 130 may be reversed.

In some examples, the heat transfer treatment device 102 can include an injection delivery system 140 for transferring an injectable material 142 and/or substance to the target area 112. The injection delivery system 140 can include one or more port(s) 144, which can be formed onto the top surface 106 and/or another surface of the heat transfer treatment device 102. In some instances, the majority or the entire top surface of the device 102 may be the port 144. The port 144 can be an opening with a rubber, self-healing port gasket for receiving a tip of an injection needle. The injectable material can include pharmaceuticals and/or medication to be delivered to the target area 112 for treatment of the tumor. For instance, the injectable material can include a chemotherapy drug, an immunotherapy drug, an anesthetic, an antibiotic, an antiviral, other biologics, chemical agents, therapeutics, and/or combinations thereof.

Furthermore, the injection delivery system 140 can include a reservoir 146 for holding the injectable material 142. The reservoir 146 can be fluidly coupled to the port 144 such that the injectable material 142 injected into the port 144 is stored in the reservoir 146. In some scenarios, the reservoir 146 is disposed directly below and/or adjacent to the port 144 at the top surface 106 of the body portion 104. Moreover, the injection delivery system 140 can include a delivery tunnel 148 fluidly coupled to the reservoir 146 and/or the port 144. The delivery tunnel 148 can include a channel formed into the body portion 104 from the port 144 and/or the reservoir 146 out the bottom surface 108. The delivery tunnel 148 can also include a tunnel extension 150 protruding from the bottom surface 108. For instance, the tunnel extension 150 of the delivery tunnel 148 can extend in a same direction as the plurality of cooling probes 116. In some instances, the device 102 may include multiple delivery tunnels 148 and a corresponding network of internal tunnels for a larger surface area of delivery of the injectable material 142. The multiple delivery tunnels may aid in distribution and/or diffusion of the injectable material 142 onto a larger surface area. The reservoir 146 can be centrally located on the top surface 106 such that the delivery tunnel 148 is also centrally located on the heat transfer treatment device 102 (e.g., from the top view perspective). Also, in this configuration, the tunnel extension 150 of the delivery tunnel 148 protruding from the bottom surface 108 can extend between and/or in the middle of the plurality of cooling probes 116. Additionally, the reservoir 146 and/or the delivery tunnel 148 can be formed of a biocompatible material.

As such, the heat transfer treatment device 102 can be a hypothermia-inducing apparatus with therapeutic delivery mechanisms formed by the injection delivery system 140. Moreover, the heat transfer treatment device 102 can be used to treat other types of tumors in addition to and/or alternatively to brain tumors. The heat transfer treatment device 102 can also be used to prevent brain tumor recurrence. Furthermore, the technology disclosed herein can be used for other disease states wherein both hypothermia and a fluid therapeutic improves treatment.

In some examples, the heat transfer treatment device 102 provides improved treatment by providing options for selectively determining a time for local injection(s) (e.g., via the injection delivery system 140) simultaneously, before, and/or after hypothermia treatment. These systems 100 can provide local hypo-, normo-, or hyper-thermia to a target area 112 with local pharmaceutical treatment. Furthermore, it is to be understood that, with or instead of the plurality of cooling probes 116, the heat transfer treatment device 102 can use a single cooling probe, a bulge, an indent, and/or other type of protrusion with a cooled surface area.

Additionally, the injection delivery system 140 can provide multiple, repeated injections of a same injectable material 142 and/or a different injectable material 142. Furthermore, the injection delivery system 140 can include a plurality of ports 144, a plurality of reservoirs 146, and/or a plurality of delivery tunnels 148, which can be used to deliver the injectable material 142 to one or multiple target locations 112. Furthermore, the system 100 can include an osmotic pump connected to the injection delivery system 140 to provide continuous/sustained pharmaceutical delivery. Moreover, in some examples of the heat transfer treatment device 102, the arrangement of the reservoir 146 and the delivery tunnel 148 can be replaced by a delivery tunnel 148 alone or an empty port 144 or a different device that provides additional fluid/biologic handling abilities (e.g., tumor biopsy/sampling) or concomitant treatment approaches. For instance, the port 144 can provide a passageway through which another device that applies local radiation or local ultrasound is passed through.

FIGS. 2A-2E depict example system(s) 100 for tumor treatment using the heat transfer treatment device 102. The system(s) 100 depicted in FIGS. 2A-2E can be similar to, identical to, and/or can from at least a portion of the system(s) depicted in FIGS. 1A and 1B.

Furthermore, the system(s) 100 shown in FIGS. 2A-2E can include multiple probe geometry designs 118 and styles for application of post-surgical therapy against brain tumors 202, and also for other intracranial applications and extracranial applications. For example, FIG. 2A illustrates a post-surgical resection cavity 202 and the surrounding areas to which the heat transfer therapy may be applied. In some instances, the region around the post-surgical resection cavity 202 (generally around 2 to 3 cm or more from the center of the cavity) is the region in which a brain tumor is likely to recut. The heat transfer therapy is generally targeted for this region, which includes intact brain matter and any residual tumor cells. These probe geometry designs 118 can interface with a thermal system 204 (e.g., including the water block layer 122, the Peltier array 130, the osmotic pump, combinations thereof, and so forth) to cool and/or apply heat to the target area 112. The probe geometry designs 118 disclosed herein are some examples of how the plurality of cooling probes 116 may be arranged to interface with the brain or other tissues. Furthermore, it is to be understood that, in some scenarios, the heat transfer treatment device 102, the probe(s) 116, and/or cooling patches disclosed herein can be implanted by hand individually or via assistance of one or more robotic devices. The heat transfer treatment device(s) 102 disclosed herein can include prefabricated arrays, assembled on-site in the surgical room, or assembled in situ or in vivo. Moreover, the disclosed probe geometry designs 118 and other designs for cooling may also be used for the additional or sole purpose of limiting injury and/or secondary injury in the brain from the implantation of electrodes, which can also be attached to the body portion 104.

FIG. 2A depicts an example system 100 with a heat transfer treatment device 102 having a first probe geometry design 206. The first probe geometry design 206 can include a platform 208 to which the plurality of cooling probes 116 are mounted, forming the multi-probe array 114). The plurality of cooling probes 116 can extend substantially parallel from the platform 208 into the target area 112 and/or a tumor in and/or proximate to the target area 112. The plurality of cooling probes 116 can be straight and/or vertical and can penetrate tissue and/or a cavity inside the patient. In some instances, the cooling probes 116 can extend further than the region of interest illustrated in FIG. 2A to ensure homogenous cooling in the region of interest.

FIG. 2B depicts an example system 100 with a heat transfer treatment device 102 having a second probe geometry design 210. The second probe geometry design 210 can include a core-and-spoke arrangement 212 in which a probe core 214 is coupled to the platform 208 via a core mounting arm 216, and the plurality of cooling probes 116 extend radially outward from the probe core 214 to facilitate heat transfer to the tissue of the target area 112. The probe core 214 and/or the core mounting arm 216 can be formed of a same material and/or a different material as the plurality of cooling probes 116 to further optimize heat transfer. In some instances, the probe core 214 may be inserted into the cavity by the core mounting arm 216 and the cooling probes 116 may be extended through an actuating device to extend away from the probe core and into the region of interest.

FIG. 2C depicts an example system 100 with a heat transfer treatment device 102 having a third probe geometry design 218. The third probe geometry design 218 can include a multi-platform arrangement 220 including a first platform 222 and a second platform 224 or any number of platforms 208. For instance, the multi-platform arrangement 220 can include the first platform 222 positioned at a surface 226 of a patient (e.g., a cranial outer surface, an epidural surface, an interior or exterior vascular wall surface, and so forth). The second platform 224 can be separated a distance from the first platform 222. The second platform 224 can be positioned below the multi-platform arrangement 220 and/or can be coupled to the first platform 222 by a platform coupler arm 228, which can extend perpendicular or at any angle from an underside or another side of the first platform 222. The second platform 224 can be operable for implantation inside the patient. The plurality of cooling probes 116 can extend from the first platform 222, the second platform 224, or both. In this arrangement, the third probe geometry design 218 can have a tiered distribution of the plurality of cooling probes 116 and/or a gap formed between the first platform 222 and the second platform 224. The platforms 222 and/or 224 can be formed of a same or different material relative to each other to control the heat transfer distribution, and/or can be formed of the same or different material as the plurality of cooling probes 116. In some examples, the second platform 224 may include one or more hinged portions that enable the platform to fold during insertion into the cavity. The hinges of the second platform 224 may also include one or more spring activators or other bias mechanisms to hold the second platform in the extended position once inserted into the cavity.

FIG. 2D depicts an example system 100 with a heat transfer treatment device 102 having a fourth probe geometry design 230. The fourth probe geometry design 230 can include a curved and/or splayed arrangement 232. For instance, one, some, most, and/or all of the plurality of cooling probes 116 can extend from the platform 208 with curves, such as radial extending curves of the splayed arrangement 232. Moreover, any of the plurality of cooling probes 116 can extend at an angle, such as multiple different angles. The splayed arrangement 232 can be used to form a spherical, hemispherical, or otherwise rounded distribution of cooling probes.

FIG. 2E depicts an example system 100 with a heat transfer treatment device 102 having a fifth probe geometry design 234. The fifth probe geometry design 234 can include a removable structural support 236 for a patch 238. For instance, the body portion 104 can include a removable layer 240 disposed at the top surface 106. The removable layer 240 can be a structural support for the heat transfer treatment device 102, used for insertion of the cooling system 128 and/or the injection delivery system 140 into the patient. Afterwards, the removable layer 240 can be peeled and/or cut away from the heat transfer treatment device 102, exposing the port 144. The removable layer 240 can form a back of the patch 238, wherein the patch includes the components of the heat transfer treatment device 102. The removable layer 240 can cover some and/or all of the heat transfer treatment device 102, and can include an adhesive, hook-and-loop, and/or magnets to assist with the removable coupling to the body portion 104. In some scenarios, the removable layer 240 also includes components of the cooling system 128 and/or the injection delivery system 140, such as additional reservoirs 146 of injectable material 142, or removable cooling probes 116 and/or a delivery tunnel 148 for changing and/or reducing a treatment dosage of the heat transfer upon removal of the removable layer 240.

It is to be understood that the different probe geometry designs 118 disclosed in FIGS. 2A-2E (e.g., the first probe geometry design 206, the second probe geometry design 210, the third probe geometry design 218, the fourth probe geometry design 230, and/or the fifth probe geometry design 234) and/or any sub-components of the first probe geometry design 206, the second probe geometry design 210, the third probe geometry design 218, the fourth probe geometry design 230, and/or the fifth probe geometry design 234 can be repeated, combined, and/or omitted. In this way the probe geometry designs 118 can be customized to deploy the plurality of cooling probes 116 to a particularly shaped target area 112 having a specific treatment region profile. This treatment region shape, which matches the probe geometry design 118, can correspond to diagnostic data of the tumor being treated, which can suggest optimal areas for cooling and/or therapeutics deployment. The probe geometry designs 118 can be further optimized by omitting the plurality of cooling probes 116 and their cooling/heat transfer activity from various other regions proximate to and/or adjacent to the target area 112, such as an area between the second platform 224 and the surface 226. These omitted areas may be determined to receive minimal or no benefit from the cooling/heat transfer activity and/or may require protection from the cooling/heat transfer activity.

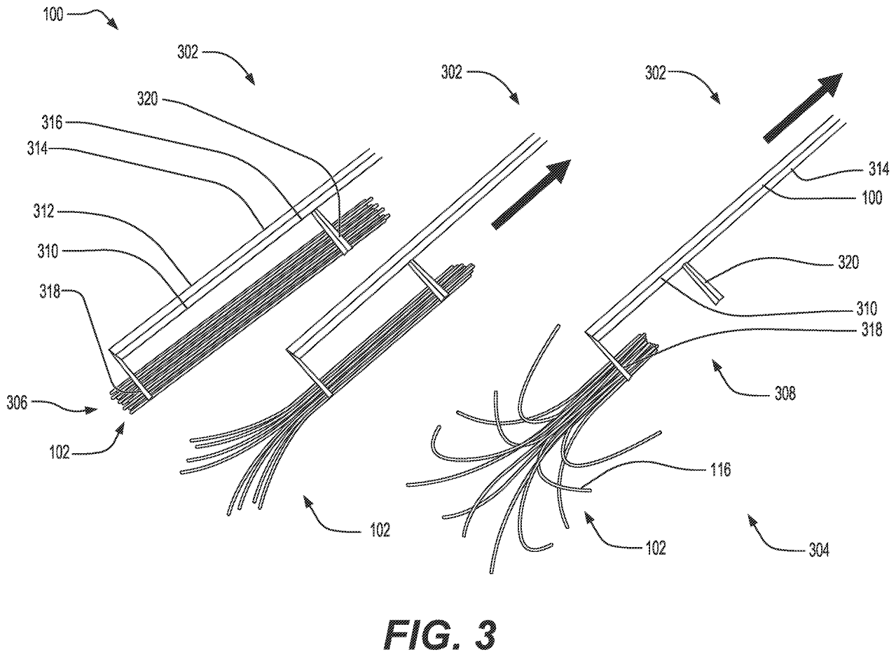

FIG. 3 depicts an example system 100 including a probe implantation device 302 for holding the plurality of cooling probes 116 during an implantation procedure 304, and/or for releasing the plurality of cooling probes 116 from an implantation configuration 306 into a heat distribution configuration 308. The system 100 depicted in FIG. 3 can be similar to, identical to, and/or can from at least a portion of the system(s) depicted in FIGS. 1A-2E.

In some examples, the probe implantation device 302 can include a first insertion rod 310 with a first elongated body 312 and/or a second insertion rod 314 with a second elongated body 316. The first insertion rod 310 can form a release sheath with a first loop 318, strap, opening, or other bundling component, and the second insertion rod 314 can form a holding sheath with a second loop 320, strap, opening, or other bundling component. The first insertion rod 310 can be slidable relative to the second insertion rod 314. Furthermore, the first loop 318 can be slidable relative to the plurality of cooling probes 116, for instance, by forming an at least somewhat loose hold on the plurality of cooling probes 116 so that the plurality of cooling probes 116 release from the first loop 318. The second loop 320 can form a tighter hold on the plurality of cooling probes 116 such that the ends of the plurality of cooling probes 116 remain held in place. Accordingly, sliding the first insertion rod 310 and/or the second insertion rod 314 can cause the first loop 318 to slide down the plurality of cooling probes 116 towards the second loop 320. This sliding action can cause the first loop 318, which was previously holding the plurality of cooling probes 116 together, to release the plurality of cooling probes 116, and the plurality of cooling probes 116 can unfold and penetrate tissue.

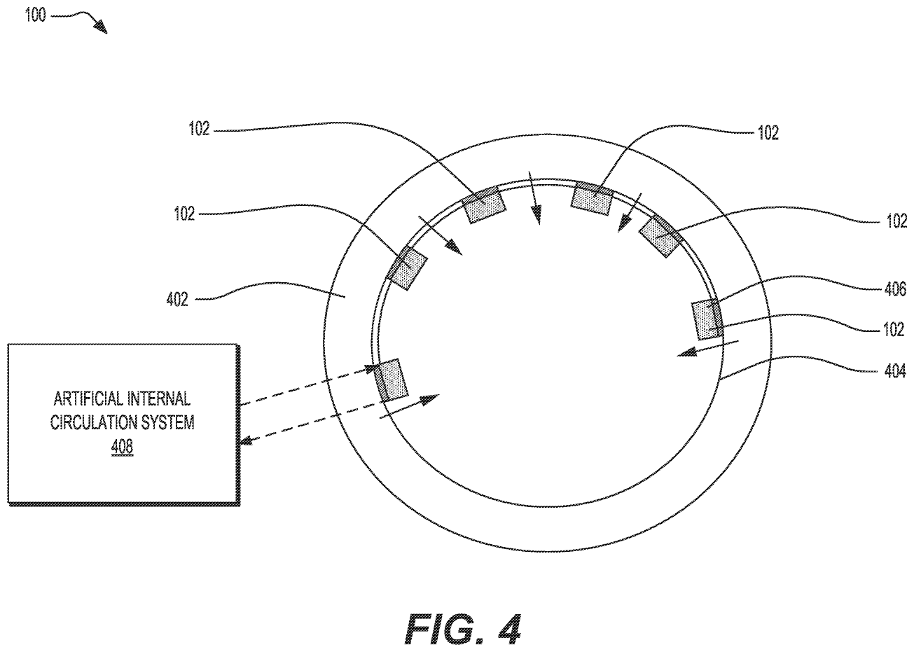

FIG. 4 depicts an example system 100 including the heat transfer treatment device 102, which includes an array of thermal systems 204 embedded in a stentrode or other stent-like device and implanted into a blood vessel 402. As shown in FIG. 4, the heat transfer treatment device 102 can be formed at a small scale for attachment at various internal body parts, including an interior wall surface 404 of the blood vessel 402.

For instance, the probes and/or devices disclosed herein can facilitate heat transfer into the blood and can be minimally invasive. One example is that of a “stentrode” that could cool a local region of tissue on one end and the heat it produces/removes is transferred to the blood with which it is in contact. In this way, the target area 112 can include a plasma and/or blood flow path within the blood vessel 402. This configuration can omit the cooling multi-probe array 114 or can include the cooling multi-probe array 114. Furthermore, the heat transfer treatment device 102 can be formed as a patch 238, as discussed above regarding FIG. 2E. As such, the heat transfer treatment device 102 can be applied to tissue surfaces (e.g., heart, kidney, joints, bone). This patch 238 form factor of the heat transfer treatment device 102 can be flexible or rigid and can include the internal structures discussed herein to facilitate cooling/heat transfer activity at these target areas 112. Furthermore, any of the components of the body portion 104, the cooling system 128, and/or the injection delivery system 140 can be made partially or entirely of biocompatible materials 406. Additionally, or alternatively, the devices and/or patches 238 disclosed herein can have some surfaces to facilitate heat transfer and/or additional separate surfaces to facilitate electrical recording and/or stimulation. Also, devices can perform all functions within the same surface (e.g., heat transfer and electrical recording/stimulation). In some scenarios, the heat transfer treatment device 102 can have a combination of the components disclosed herein for recording and/or stimulating the cortical layers of the brain while applying heat transfer via cooling or heating to the deeper white matter structures of the brain.

Moreover, the system(s) 100 disclosed herein can include the thermal system 204 for facilitating thermal management of tissues, implants, or bionics by transferring or distributing heat to other parts of the body. Some systems of heat management can use percutaneous components to pump heat directly out of the body. These methods can transfer heat directly to the air outside the body or to an external refrigerator or cooler or cooling system. However, these methods may use either percutaneous components (e.g., which means components that pass through the skin) or, more generally, some connection between the inside of the body (e.g., anywhere beneath the skin) to outside the body (e.g., outside the skin). This can result in a nidus for infections at the least, and also improper healing and implant/device/transplant damage. Instead, some systems disclosed herein include full implantation wherein there is no direct communication between the inside and outside of the body.

For instance, the thermal system 204 can include one or more components of an artificial internal circulation system 408 which manages heat from components or tissues inside the body by transferring it to other locations inside the body. This can be accomplished via any combination of a pump, heat sinks, water blocks, vapor chambers, and tubing to facilitate the distribution of heat. Such a system could be attached or applied to tissue, implanted devices, or bionics that require temperature management, in addition to or alternatively to the heat transfer treatment device 102. In some instances, the artificial internal circulation system 408 can distribute the heat around the body such that both the source and sink of the heat do not reach temperatures upon which tissue damage could eventually occur (e.g., maintaining temperature to be less than 40° C. continuously or brief spurts of increased temperature for reduced amounts of time, such as less than 60° C. for 1 hour).

The artificial internal circulation system 408 can perform temperature management of tissue, implanted devices (e.g., the heat transfer treatment device 102), and/or bionics. The artificial internal circulation system 408 can include any combination of a pump, heat sinks, water blocks, vapor chambers that remain within the body, and can be used to remove/manage heat from implanted devices. This can facilitate the use of high-power implants/computer chips. For instance, the artificial internal circulation system 408 can be used on any implant in the body that uses heat dissipation to remove and/or manage heat from tissues. The artificial internal circulation system 408 can also be used for any method of heat dispersion that fully remains within the body or under the skin. In some iterations, there could be percutaneous components to power the system; however, the primary purpose of these percutaneous components can be something other than heat transfer. In other iterations, concomitant use of transcutaneous energy transfer systems can obviate the need for any percutaneous components to power the system. The artificial internal circulation system 408 can be used with a method of heat dispersion that primarily remains within the body or under the skin but has supplemental heat dispersion via a percutaneous component. Furthermore, the artificial internal circulation system 408 can be used to transfer heat towards the bloodstream and/or can use the bloodstream to keep certain parts at body temperature.

Furthermore, the angiogenic factors can be used by the artificial internal circulation system 408 to increase blood flow to facilitate keeping the device at body temperature. Moreover, components of the artificial internal circulation system 408 can be implanted at any location in the body including the skin, under the skin, within muscles, within body cavities, and so forth. Use of one or more heatsinks, water blocks, and/or vapor chambers to enable heat distribution can include implanting tubing of various lengths to carry fluid and/or coolant to any location within the body. Passive convection management can be provided without a water block in some scenarios, for instance, by using a heat sink or vapor chamber instead.

Additionally, the artificial internal circulation system 408 can use external devices to facilitate temperature management (e.g., a helmet to provide scalp cooling). External components can also include a battery or power source, a harness, an external heat sink, and so forth. Also, the artificial internal circulation system 408 can include wireless power and/or wired power provided to the system. Moreover, the artificial internal circulation system 408 can include a water block and/or heat sink with any geometry or form factor. The artificial internal circulation system 408 can be easily removable, replaceable, and/or upgradeable. Moreover, the water block can be microfluidic and fabricated using microfluidics. Also, the water block can be a multilayered water block. Flow rates for the water block and/or the water block layer 122 can be less than 1 ml/s or greater than 1 ml/s but less than 10 ml/s or greater than 10 ml/s but less than 100 ml/s. Additionally, the manufacturing procedure for any components of the heat transfer treatment device 102 and/or the artificial internal circulation system 408, such as the water block, can be conducted via 3D printing or standard additive or subtractive manufacturing methods.

In some scenarios involving cooling tissue or tumors via one or more probes (e.g., the plurality of cooling probes 116), the ambient temperature of the target area 112 can be brought below body temperature. The devices, parameters, and methods to facilitate cooling can be used with or without simultaneous electrical recording and/or stimulation. For instance, hypothermia/cooling using any of the techniques disclosed herein can be used to reduce or prevent electrode implantation-induced injury, for instance, in scenarios using brain machine interfaces or peripheral nerve interfaces or spinal cord interfaces. Hypothermia/cooling can reduce scarring from chronic penetrating or surface electrode implantation. This can be used to facilitate recording (e.g., from brain machine interfaces) for longer periods. The thermal system 204 and/or the artificial internal circulation system 408 can provide a paradigm of cooling (e.g., an “on-off” schedule or profile) to balance electrical recording or stimulation and can limit brain tissue injury or scarring.

In some examples, the 204/and/or the artificial internal circulation system 408 can be used to create hypothermic temperatures that selectively kill tumor cells in the range of 15-35° C. The artificial internal circulation system 408 and/or the cooling multi-probe array 114 can also be used to create hypothermic temperatures that stop tumor growth at <20° C. Additionally, the artificial internal circulation system 408 and/or the cooling multi-probe array 114 can be used to treat tissue with or without remnant tumor cells.

Furthermore, the artificial internal circulation system 408 and/or the cooling multi-probe array 114 can use microwires or micrometer/submillimeter thin probes, for instance, to form the plurality of cooling probes 116. The plurality of cooling probes 116 can have varying thickness along their lengths and/or with respect to each other. Furthermore, the system 100 can use robotic placement of cooling probes 116, whether superficial or deep. The system 100 can provide simultaneous cooling with, before, and/or after providing heating, electrical recording, electrical stimulating, and/or treatment. For instance, the system 100 can include different, individual probes for different purposes, such as cooling, heating, recording, stimulating, and/or treating. The probes (e.g., the plurality of cooling probes 116) can be integrated with or separate from temperature sensors. Moreover, the system 100 can provide any duration and/or intermittency of treatment, including complicated paradigms, and the various components disclosed herein can be removable, replaceable, and/or upgradeable.

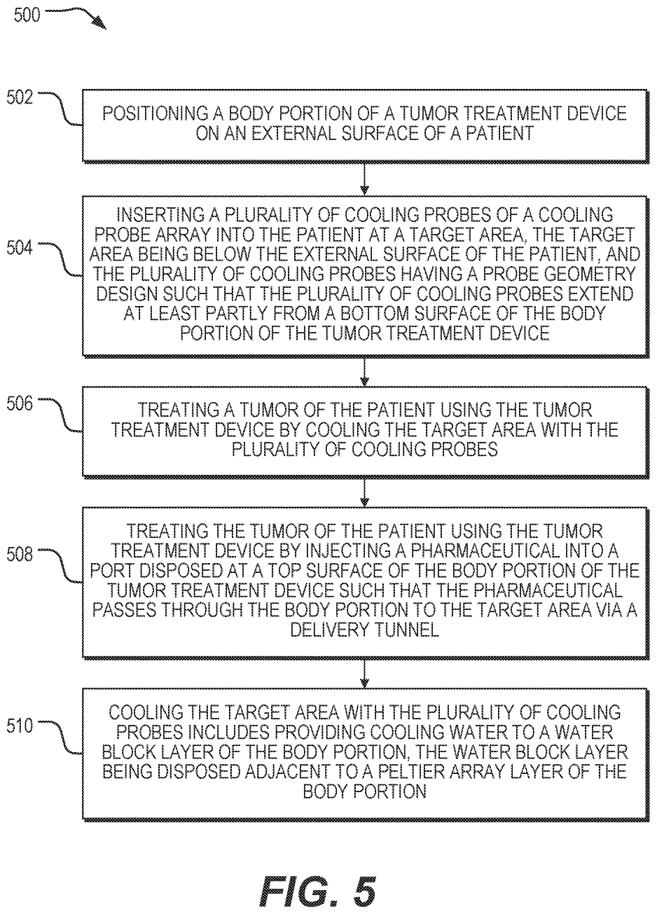

Turning to FIG. 5, an example method(s) 500 of treating a patient with a heat transfer treatment device 102 is depicted. The method(s) 500 of FIG. 5 can be performed by the system(s) 100 discussed above regarding FIGS. 1A-4.

In some examples, at operation 502, the method 500 can position a body portion of a tumor treatment device on an external surface of a patient. At operation 504, the method 500 can insert a plurality of cooling probes of a cooling probe array into the patient at a target area, the target area being below the external surface of the patient, and the plurality of cooling probes can have a probe geometry design such that the plurality of cooling probes extends at least partly from a bottom surface of the body portion of the tumor treatment device. At operation 506, the method 500 can treat a tumor of the patient using the tumor treatment device by cooling the target area with the plurality of cooling probes. At operation 508, the method 500 can treat the tumor of the patient using the tumor treatment device by injecting a pharmaceutical into a port disposed at a top surface of the body portion of the tumor treatment device such that the pharmaceutical passes through the body portion to the target area via a delivery tunnel. At operation 510, the method 500 can cool the target area with the plurality of cooling probes by a Peltier array layer of the body portion. In particular, the Peltier elements transfer heat from the tissue and accumulates the transferred heat onto a “hot side” of the element. The hot side of the Peltier elements may be cooled through circulation of cooling water to the water block layer of the body portion disposed adjacent to the Peltier array layer.

It is to be understood that the specific order or hierarchy of steps in the method(s) depicted in FIG. 5 and throughout this disclosure are instances of example approaches and can be rearranged while remaining within the disclosed subject matter for treating remnant tumor cells that may have infiltrated brain tissue in the region of interest. For instance, any of the operations depicted in FIG. 5 and throughout this disclosure may be omitted, repeated, performed in parallel, performed in a different order, and/or combined with any other of the operations depicted in FIG. 5 and throughout this disclosure.

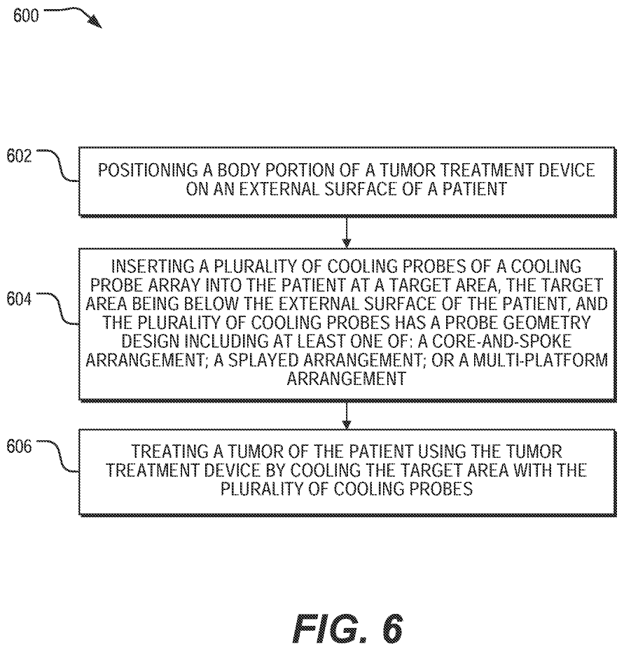

Turning to FIG. 6, an example method(s) 600 of treating a patient with a heat transfer treatment device 102 is depicted. The method(s) 600 of FIG. 5 can be performed by the system(s) 100 discussed above regarding FIGS. 1A-4.

In some examples, at operation 602, the method 600 can position a body portion of a tumor treatment device on an external surface of a patient. At operation 604, the method 600 can insert a plurality of cooling probes of a cooling probe array into the patient at a target area, the target area being below the external surface of the patient, and the plurality of cooling probes can have a probe geometry design including at least one of a core-and-spoke arrangement; a splayed arrangement; or a multi-platform arrangement. At operation 606, the method 600 can treat a tumor of the patient using the tumor treatment device by cooling the target area with the plurality of cooling probes.

It is to be understood that the specific order or hierarchy of steps in the method(s) depicted in FIG. 6 and throughout this disclosure are instances of example approaches and can be rearranged while remaining within the disclosed subject matter. For instance, any of the operations depicted in FIG. 6 and throughout this disclosure may be omitted, repeated, performed in parallel, performed in a different order, and/or combined with any other of the operations depicted in FIG. 6 and throughout this disclosure.

While the presently disclosed technology has been described with reference to various implementations, it will be understood that these implementations are illustrative and that the scope of the presently disclosed technology is not limited to them. Many variations, modifications, additions, and improvements are possible. More generally, implementations in accordance with the presently disclosed technology have been described in the context of particular implementations. Functionality may be separated or combined differently in various implementations of the disclosure or described with different terminology. These and other variations, modifications, additions, and improvements may fall within the scope of the disclosure.

What is claimed is:

1. A tumor treatment device comprising:
a body portion having a top surface and a bottom surface; and
a cooling probe array with a probe geometry design such that a plurality of cooling probes extend at least partly from the bottom surface.

2. The tumor treatment device of claim 1, further comprising:
an injection delivery system operable to provide an injectable substance through the body portion to a target area.

3. The tumor treatment device of claim 1, the bottom surface comprising:
a water block layer; and
a Peltier array disposed adjacent to the water block layer.

4. A tumor treatment device comprising:
a body portion having a top surface and a bottom surface;
a cooling probe array with a probe geometry design such that a plurality of cooling probes extend at least partly from the bottom surface; and
an injection delivery system operable to provide an injectable substance through the body portion to a target area.

5. The tumor treatment device of claim 4, wherein the body portion includes a water block layer defining the top surface.

6. The tumor treatment device of claim 5, wherein the injection delivery system includes a reservoir operable to hold the injectable substance being provided to the target area.

7. The tumor treatment device of claim 6, wherein, the reservoir is formed on the water block layer at the top surface.

8. The tumor treatment device of claim 6, wherein the reservoir is located on the top surface.

9. The tumor treatment device of claim 6, wherein the injection delivery system includes a delivery tunnel passing at least partly through the body portion to provide the injectable substance to the target area.

10. The tumor treatment device of claim 9, wherein the delivery tunnel extends out the bottom surface.

11. The tumor treatment device of claim 9, wherein the delivery tunnel includes an extension portion protruding from the bottom surface between the plurality of cooling probes.

12. The tumor treatment device of claim 9, wherein the reservoir and the delivery tunnel are formed of biocompatible material.

13. The tumor treatment device of claim 12, wherein the injection delivery system includes a port disposed at the top surface operable to receive a needle providing the injectable substance.

14. The tumor treatment device of claim 13, wherein the port is fluidly coupled to the delivery tunnel.

15. The tumor treatment device of claim 13, wherein the port is fluidly coupled to the reservoir.

16. The tumor treatment device of claim 5, wherein the body portion includes a Peltier array layer disposed between the water block layer and the cooling probe array.

17. A tumor treatment system comprising:
a body portion of a tumor treatment device having a top surface and a bottom surface;
a cooling probe array of the tumor treatment device having a probe geometry design such that a plurality of cooling probes extend at least partly from the bottom surface, the cooling probe array being operable to cool a target area of a patient; and
an injection delivery system operable to provide an injectable substance through the body portion.

18. The tumor treatment system of claim 17, wherein the injection delivery system includes:
a port operable to receive the injectable substance;
a reservoir fluidly coupled to the port; and
a delivery tunnel fluidly coupled to the reservoir and extending through the body portion to the target area.

19. The tumor treatment system of claim 17, wherein
the body portion is operable for placement at an interior wall of a blood vessel; and
the target area includes a plasma flow path within the blood vessel.

20. A method of tumor treatment, the method comprising:
positioning a body portion of a tumor treatment device on an external surface of a patient;
inserting a plurality of cooling probes of a cooling probe array into the patient at a target area, the target area being below the external surface of the patient, and the plurality of cooling probes having a probe geometry design such that the plurality of cooling probes extend at least partly from a bottom surface of the body portion of the tumor treatment device; and
treating a region near a tumor of the patient using the tumor treatment device by cooling the target area with the plurality of cooling probes.

21. The method of claim 21, wherein treating the tumor of the patient using the tumor treatment device further includes injecting a pharmaceutical into a port disposed at a top surface of the body portion of the tumor treatment device such that the pharmaceutical passes through the body portion to the target area via a delivery tunnel.

22. The method of claim 21, wherein cooling the target area with the plurality of cooling probes includes providing cooling water to a water block layer of the body portion, the water block layer being disposed adjacent to a Peltier array layer of the body portion.

23. The method of claim 21, wherein treating the tumor of the patient includes transferring a therapeutic from a reservoir disposed in the body portion to the target area simultaneously with cooling the target area.

24. A tumor treatment device comprising:
a body portion having a top surface and a bottom surface, the body portion including:
a water block layer; and
a Peltier array disposed adjacent to the water block layer; and
a cooling probe array with a probe geometry design such that a plurality of cooling probes extend at least partly from the bottom surface.

25. The tumor treatment device of claim 24, wherein the probe geometry design includes a probe mounting platform, and at least some of the plurality of cooling probes extend perpendicularly from the probe mounting platform.

26. The tumor treatment device of claim 24, wherein the probe geometry design includes at least a core-and-spoke arrangement of the plurality of cooling probes.

27. The tumor treatment device of claim 24, wherein the probe geometry design includes a plurality of curved probes forming a splayed arrangement.

28. The tumor treatment device of claim 24, wherein the probe geometry design includes a patch with a removable structural support.

29. The tumor treatment device of claim 28, wherein the removable structural support has a first configuration as a layer on the top surface and a second configuration removed from the top surface.

30. The tumor treatment device of claim 28, wherein an injection delivery system is exposed upon removing the removable structural support.

31. The tumor treatment device of claim 24, wherein the probe geometry design includes a multi-platform arrangement.

32. The tumor treatment device of claim 31, wherein the multi-platform arrangement includes at least:
a first probe mounting platform operable for placement on an outer cranial surface; and
a second probe mounting platform operable for placement under the outer cranial surface.

33. The tumor treatment device of claim 32, wherein the first probe mounting platform is coupled to the second probe mounting platform by a platform extension arm.

34. The tumor treatment device of claim 33, wherein the platform extension arm couples to a bottom surface of the first probe mounting platform and a top surface of the second probe mounting platform.

35. The tumor treatment device of claim 24, wherein the plurality of cooling probes includes one or more first cooling probes extending from the first probe mounting platform and one or more second cooling probes extending form the second probe mounting platform.

36. The tumor treatment device of claim 24, wherein the water block layer defines the top surface of the body portion.

37. The tumor treatment device of claim 24, wherein the Peltier array causes a heat transfer between the water block layer and the cooling probe array.

38. A tumor treatment system comprising:
a body portion of a tumor treatment device having a top surface and a bottom surface;
cooling portion of the top surface in fluid communication with a water block; and
a cooling probe array of the tumor treatment device having a probe geometry design such that a plurality of cooling probes extend at least partly from a bottom surface of the body portion, the probe geometry design being operable to cool a target area of a patient, and the probe geometry design including at least one of:
a core-and-spoke arrangement;
a splayed arrangement;
a multi-platform arrangement; or
a removable structural support.

39. The tumor treatment system of claim 38, further comprising:
a probe implantation device including a release sheath operable to maintain the plurality of cooling probes in a first position and release the plurality of cooling probes into a second position.

40. The tumor treatment system of claim 38, wherein the first position is a bundled position and the second position is a splayed position.

41. A method of tumor treatment, the method comprising:
positioning a body portion of a tumor treatment device on an external surface of a patient;
inserting a plurality of cooling probes of a cooling probe array into the patient at a target area, the target area being below the external surface of the patient, and the plurality of cooling probes has a probe geometry design including at least one of:
a core-and-spoke arrangement;
a splayed arrangement; or
a multi-platform arrangement; and
treating a tumor of the patient using the tumor treatment device by cooling the target area with the plurality of cooling probes.

42. The method of claim 41, wherein inserting the plurality of cooling probes into the patient includes:
inserting a probe implantation device into the patient; and
sliding a release sheath of the probe implantation device out of the patient.

43. The method of claim 41, wherein positioning the body portion of the tumor treatment device includes:
securing the body portion to the external surface of the patient using a removal structural support disposed on a top surface of the body portion; and
removing the removal structural support from the body portion.
